(19) United States
(12) Patent Application Publication
(10) Pub. No.: US 2025/0318978 A1
(43) Pub. Date: Oct. 16, 2025

(54) APPARATUS AND METHOD FOR FORESTALLING AND PREVENTING GAIT FREEZING

(71) Applicant: Salted Co., Ltd., Pohang (KR)

(72) Inventors: Kyung Hoon KANG, Seoul (KR); Hyung Jin CHO, Seoul (KR)

(21) Appl. No.: 18/640,010

(22) Filed: Apr. 19, 2024

(30) Foreign Application Priority Data
Apr. 12, 2024 (KR) 10-2024-0049644

(51) Int. Cl. A61H 3/00; A43B 3/44; A61H 23/00

(52) U.S. Cl. CPC A61H 3/00; A43B 3/44; A61H 23/00; A61H 2003/007; A61H 2201/1642; A61H 2201/165; A61H 2201/5071; A61H 2201/5084; A61H 2205/12; A61H 2230/625

(57) ABSTRACT

The present disclosure discloses an apparatus and method for forestalling and preventing freezing of gait using a smart insole comprising sensors and a vibratory apparatus. An apparatus for forestalling and preventing freezing of gait comprises a sensing unit comprising at least one sensor; a vibration unit comprising at least one vibration generator; and a controller connected with the sensing unit and the vibration unit, wherein the controller receives from the sensing unit measurement values of the at least one sensor to determine whether freezing of gait occurs and controls the vibration unit to generate vibrations using the at least one vibration generator when it is determined as freezing of gait.

### CROSS REFERENCE TO RELATED APPLICATION

This application claims priority to Korea Patent Application No. 10-2024-0049644 filed on Apr. 12, 2024, which is hereby incorporated by reference in its entirety.

### BACKGROUND

### 1. Field of Technology

The present disclosure relates to a technology for forestalling and preventing freezing of gait. In particular, it relates to an apparatus and method for forestalling and preventing freezing of gait using a smart insole comprising sensors and a vibratory apparatus.

### 2. Related Technology

Recently, there have been various research efforts on a technology for measuring users' postures and exercise states using smart insoles equipped with sensors. Additionally, various products based on this technology, which can be utilized for exercise assistance, rehabilitation, and treatment are being developed.

Freezing of gait (FoG) is a motor symptom occurring in patients with Parkinson's disease, which includes bradykinesia, resting tremors, rigidity, and postural instability. Patients with Parkinson's disease frequently fall due to the freezing of gait, which may increase the risk of bone fractures. In this term, the freezing of gait is a main factor affecting the everyday lives or quality of lives of these patients.

Smart insoles may help in preventing and resolving freezing of gait by detecting abnormal motions caused by freezing of gait and stimulating a user with vibrations.

The discussions in this section are only to provide background information and do not constitute an admission of prior art.

### SUMMARY

The present disclosure is to provide an apparatus and method for forestalling and preventing freezing of gait using a smart insole equipped with sensors and a vibratory apparatus.

An aspect of the present disclosure provides an apparatus for forestalling and preventing freezing of gait, comprising: a sensing unit comprising at least one sensor; a vibration unit comprising at least one vibration generator; and a controller connected with the sensing unit and the vibration unit, wherein the controller receives from the sensing unit measurement values of the at least one sensor to determine freezing of gait and controls the vibration unit to generate vibrations using the at least one vibration generator when it is determined as freezing of gait.

The sensing unit may comprise at least one of a pressure sensor and an inertial measurement unit. The inertial measurement unit may be referred to as IMU.

The controller may determine based on measurement values of the pressure sensor whether a user's foot is in contact with the ground or separates from the ground.

The controller may obtain movement information of a user's foot based on measurement values of the inertial measurement unit and determine freezing of gait based on at least one of the size, period, tempo, direction, and duration of a movement.

The controller may apply different criteria for determining freezing of gait depending on whether both of a user's feet are in contact with the ground or one of the user's feet separates from the ground.

When both of a user's feet are in contact with the ground, the controller may control the vibration unit to generate vibrations after a predetermined time has passed regardless of determination of freezing of gait.

When the duration, during which both of a user's feet are in contact with the ground, has exceeded a predetermined time and vibrations have been generated, the controller may determine whether to continue generating vibrations based on a specific motion of the user.

When the specific motion of the user is detected after the duration, during which both of a user's feet are in contact with the ground, has exceeded a predetermined time and vibrations have been generated, the controller may stop generating vibrations. When the specific motion of the user is not detected after the duration, during which both of the user's feet are in contact with the ground, has exceeded a predetermined time and vibrations have been generated, the controller may continue generating vibrations.

When one of the user's feet separates from the ground and at least one of the size, period, tempo, direction, and duration of a movement of the foot separating from the ground is within a predetermined range, the controller may determine it as freezing of gait.

The controller may calculate a stride or a step speed based on at least one of the size, period, tempo, direction, and duration of a movement. When the stride or step speed is less than at least one of an average stride and an average step speed during a predetermined time, the controller may determine it as a precursor state of freezing of gait and generate vibrations.

Another aspect of the present disclosure provides a method for forestalling and preventing freezing of gait, which is performed in a computing device equipped with at least one processor and a memory for storing at least one program to be executed by the at least one processor, comprising: determining freezing of gait by receiving measurement values from at least one sensor; and generating vibrations by controlling at least one vibration generator when it is determined as freezing of gait.

In determining freezing of gait, whether a user's foot is in contact with the ground or separates from the ground may be determined based on measurement values of a pressure sensor.

In determining freezing of gait, movement information of a user's foot may be obtained based on measurement values of an inertial measurement unit and freezing of gait may be determined based on at least one of the size, period, tempo, direction, and duration of a movement.

In determining freezing of gait, different criteria for determining freezing of gait may be applied depending on whether both of a user's feet are in contact with the ground or one of the user's feet separates from the ground.

In generating vibrations, when both of a user's feet are in contact with the ground, vibrations may be generated by controlling the at least one vibration generator after a predetermined time has passed regardless of determination of freezing of gait.

In generating vibrations, when the duration, during which both of a user's feet are in contact with the ground, has exceeded a predetermined time and vibrations have been generated, whether to continue generating vibrations may be determined based on a specific motion of the user.

In generating vibrations, when the specific motion of the user is detected after the duration, during which both of the user's feet are in contact with the ground, has exceeded a predetermined time and vibrations have been generated, the vibrations may stop being generated, and when the specific motion of the user is not detected after the duration, during which both of the user's feet are in contact with the ground, has exceeded a predetermined time and vibrations have been generated, the vibrations may continue being generated.

In determining freezing of gait, when one of a user's feet separates from the ground and at least one of the size, period, tempo, direction, and duration of a movement of the foot separating from the ground is within a predetermined range, it may be determined as freezing of gait.

In determining freezing of gait, a stride or a step speed may be calculated based on at least one of the size, period, tempo, direction, and duration of the movement, and when the stride or step speed is less than at least one of an average stride and an average step speed during a predetermined time, it may be determined as a precursor state of freezing of gait and vibrations may be generated.

Embodiments of the present disclosure allow the detection and prediction of freezing of gait, and the prevention and resolution of freezing of gait by delivering stimulation to a user through vibrations.

### BRIEF DESCRIPTION OF THE DRAWINGS

In order that the disclosure may be well understood, there will now be described various forms thereof, given by way of example, reference being made to the accompanying drawings, in which:

FIG. 1 is a configuration diagram of an apparatus for forestalling and preventing freezing of gait according to an embodiment;

FIGS. 2A, 2B, 3, 4, 5 and 6 are exemplary diagrams illustrating operations of an apparatus for forestalling and preventing freezing of gait according to an embodiment;

FIG. 7 is an exemplary diagram illustrating flows of an operation of an apparatus for forestalling and preventing freezing of gait according to an embodiment; and

FIG. 8 is a flow diagram illustrating a method for forestalling and preventing freezing of gait according to an embodiment.

### DETAILED DESCRIPTION OF EMBODIMENTS

Embodiments of the present disclosure will now be described in detail in reference to the accompanying drawings. In describing the present disclosure, a detailed description of a well-known configuration or function related the present disclosure, which may obscure the subject matter of the present disclosure, will be omitted. Further, terms described below are defined in consideration of the functions of the present disclosure, and may have different meanings according to the intentions of a user or operator or the practices. Therefore, its definition will be made based on the overall contents of this specification.

Embodiments of an apparatus and method for forestalling and preventing freezing of gait will now be described in detail in reference to the accompanying drawings.

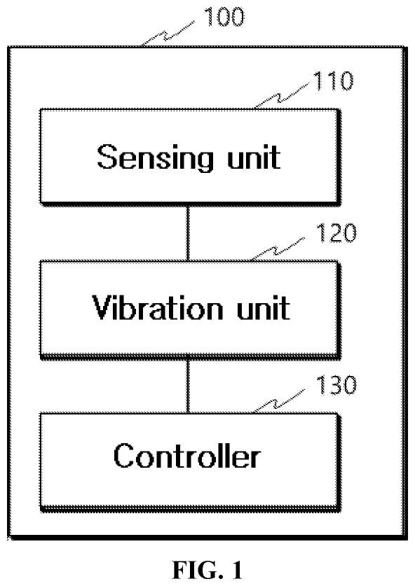

FIG. 1 is a configuration diagram of an apparatus for forestalling and preventing freezing of gait according to an embodiment.

According to an embodiment, the apparatus for forestalling and preventing freezing of gait may comprise a sensing unit 110 comprising at least one sensor, a vibration unit 120 comprising at least one vibration generator, and a controller 130 connected with the sensing unit 110 and the vibration unit 120.

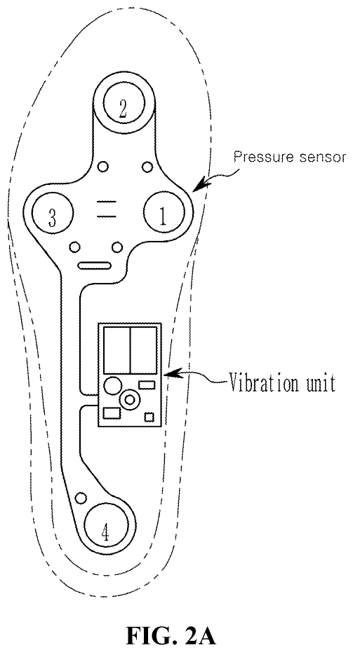

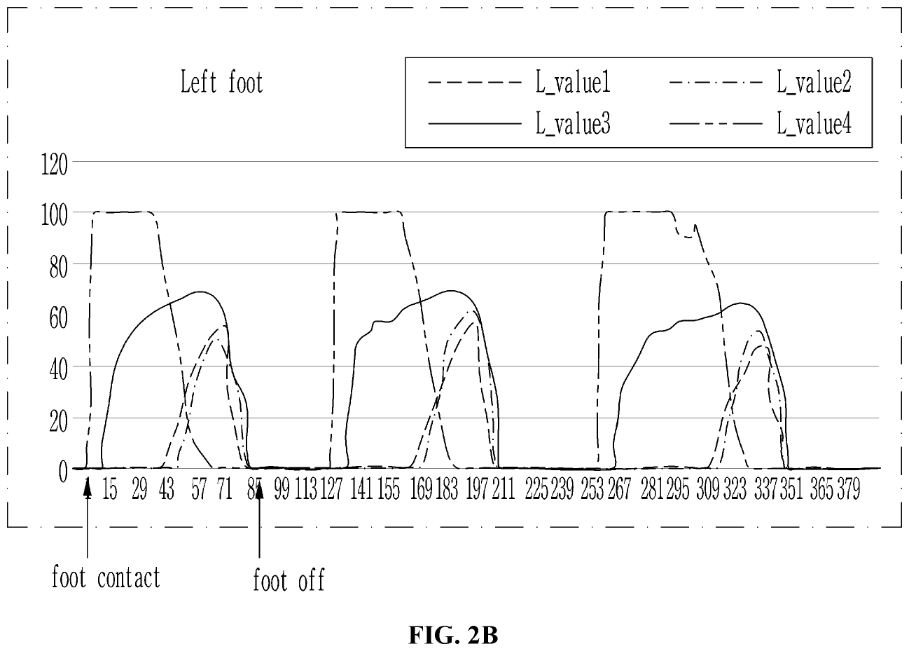

According to an example, the sensing unit 110 may include at least one of a pressure sensor and an inertial measurement unit. As an example, the sensing unit 110 may be included in an insole. Referring to FIG. 2A, the sensing unit 110 may comprise one or more pressure sensors 1˜4. These pressure sensors enable the sensing unit 110 to measure pressures corresponding to a user's respective steps. As an example, the sensing unit 110 may include at least one inertial measurement unit. This inertial measurement unit enables the sensing unit 110 to measure the direction, tempo, and acceleration of a movement of a foot, and this allows measuring at least one of the size, period, tempo, direction, and duration of the movement.

As an example, the sensing unit 110 may be provided in an insole of one foot or in insoles of two feet of a user to measure various values.

According to an example, the vibration unit 120 may be comprised in an insole as shown in FIG. 2A and generate vibrations according to control of the controller 130 to stimulate a sole of a user.

According to an embodiment, the controller 130 may determine freezing of gait by receiving at least one measurement value from the sensing unit 110. As an example, the controller 130 may measure gaits or postures of a user using signals received from various sensors and this allows the controller 130 to determine whether the user is in a state of freezing of gait or to predict occurrence of freezing of gait.

According to an embodiment, the controller 130 may determine whether a user's foot is in contact with the ground or separates from the ground based on measurement values of the pressure sensors. For instance, as shown in FIG. 2B, the controller 130 may determine whether a user's foot is in contact with the ground or separates from the ground when using pressure sensors. Further, the controller 130 may calculate the speed and the right and left balance of steps based on the measurement values of the pressure sensors.

According to an embodiment, the controller 130 may obtain movement information of a user's feet based on measurement values of the inertial measurement unit and determine freezing of gait based on the size, period, tempo, direction, and duration of a movement.

As an example, the inertial measurement unit may measure the direction, speed, and acceleration of a movement of a foot. This enables the controller 130 to calculate a stride or a step speed or calculate at least one of the size, period, tempo, direction, and duration of a foot movement. For example, the size of a movement may include the amplitude of a swinging foot, the height to which a foot is lifted, the width of a step, etc. The period of a movement may include the period of a step or the period of a foot swing. The tempo of a movement may include the speed of a movement or a swing of a foot. The direction may include the direction of a step or the direction of a foot swing. The duration, which indicates a duration during which a specific movement lasts, may include a duration, during which a foot moves or does not move, or a duration of a swing of a foot.

For example, when freezing of gait occurs, vibrations from foot movements different from those of a normal gait may be generated. The controller 130 may determine or predict freezing of gait by detecting at least one of the movements of a certain gait and the vibrations different from the movements of a normal gait.

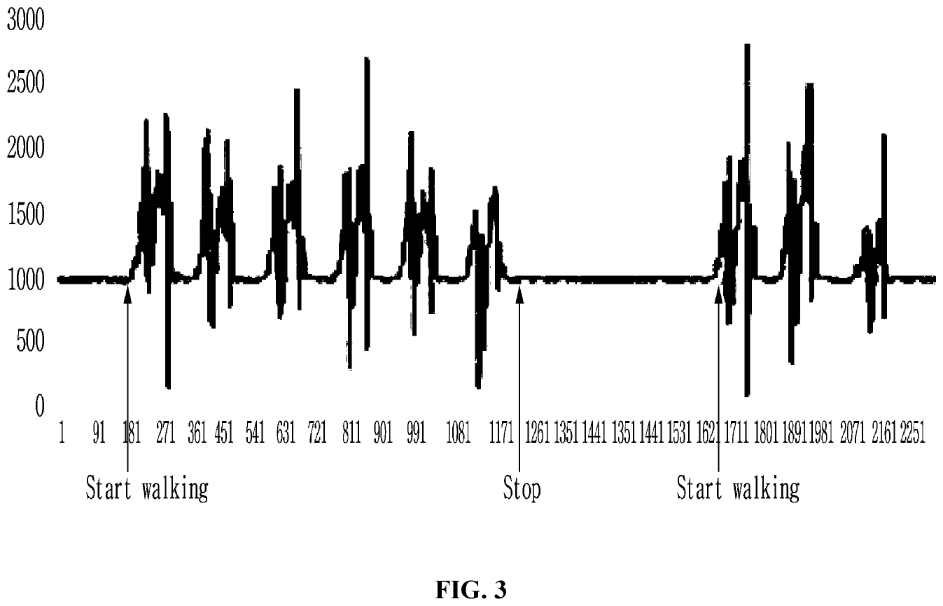

Referring to FIG. 3, when a user walks normally, movements having a predetermined amplitude may be measured. On the contrary, when the user stands in one place, no movement may be detected. For example, when no movement is detected in a state where a pressure equal to or higher than a predetermined level is measured by the pressure sensor, the controller 130 may determine that a user stands. On the contrary, when a value measured by the pressure sensor is equal to or lower than a predetermined level and no movement is detected, the controller 130 may determine that a user does not wear shoes or is seated with two feet floating.

According to an embodiment, the controller 130 may apply different criteria for determining freezing of gait depending on whether both of a user's feet are in contact with the ground or one of the user's feet separates from the ground.

For example, as shown in FIG. 3, when a user stops with both feet in contact with the ground, no movement may be detected. However, in a state where both feet are in contact with the ground, even when freezing of gait occurs, vibrations may not be generated due to the gravity and the stability from the contact of the feet with the ground. Unlike this, when freezing of gait occurs in a state where one foot is not in contact with the ground, vibrations may be generated in the floating foot. Accordingly, the controller 130 needs to be able to determine or predict freezing of gait even when no vibration is detected in a state where a user's both feet are in contact with the ground.

According to an embodiment, when a predetermined time has passed in a state where both feet of a user are in contact with the ground, the controller 130 may determine that freezing of gait occurs. For another example, when a predetermined time has passed in a state where both feet of a user are in contact with the ground, the controller 130 may control the vibration unit to generate vibrations regardless of the occurrence of freezing of gait. The reason is that, when both feet are in contact with the ground, vibrations or movements may not be detected even if freezing of gait occurs.

According to an embodiment, when the controller 130 generates vibrations based on the lapse of a predetermined time where both feet of a user are in contact with the ground, the controller 130 may determine whether to continue generating vibrations based on a specific movement of a user.

For example, when a predetermined time has passed in a state where both feet of a user are in contact with the ground, the controller 130 may determine it as the occurrence of freezing of gait and generate vibrations, or the controller 130 generates vibrations regardless of the state, even when that is not a case where freezing of gait actually occurs.

According to an embodiment, the controller 130 may stop generating vibrations when a specific movement is detected after the controller 130 has generated vibrations based on the lapse of a predetermined time where both feet of a user are in contact with the ground. For example, when a user does a specific movement after the vibrations have generated, the controller 130 may re-determine that it is not freezing of gait and stop generating vibrations. For instance, when a user performs a predetermined movement, such as moving or stamping its feet, the controller 130 may stop generating vibrations.

According to an embodiment, when a specific movement is not detected after the controller 130 has generated vibrations based on the lapse of a predetermined time where both feet of a user are in contact with the ground, the controller 130 may continue generating vibrations. For example, when any specific movements of a user are not detected even after the generation of vibrations, the controller 130 may continue generating vibrations.

According to an embodiment, when one foot of a user separates from the ground and at least one of the size, period, tempo, direction, and duration of a movement of the foot separated from the ground is within a predetermined range, the controller 130 may determine it as freezing of gait.

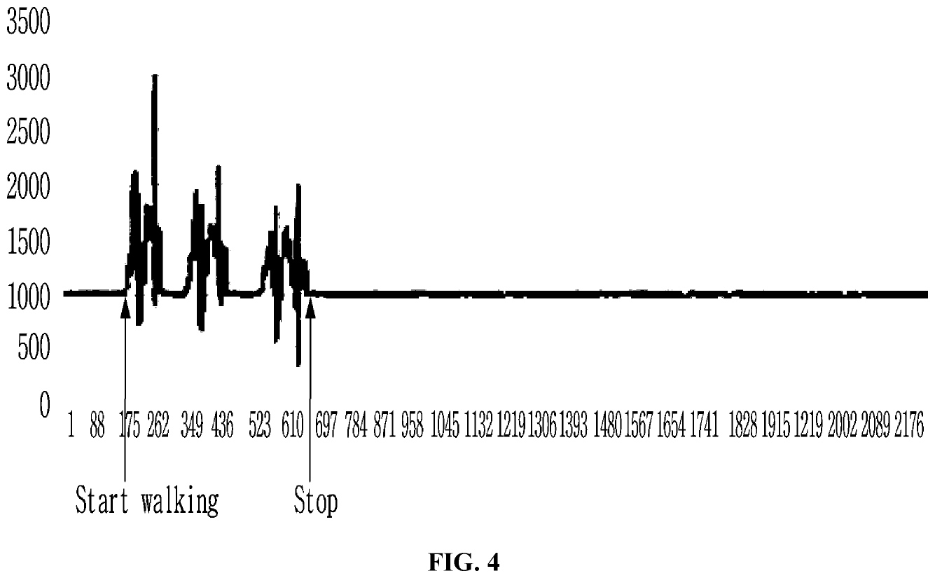

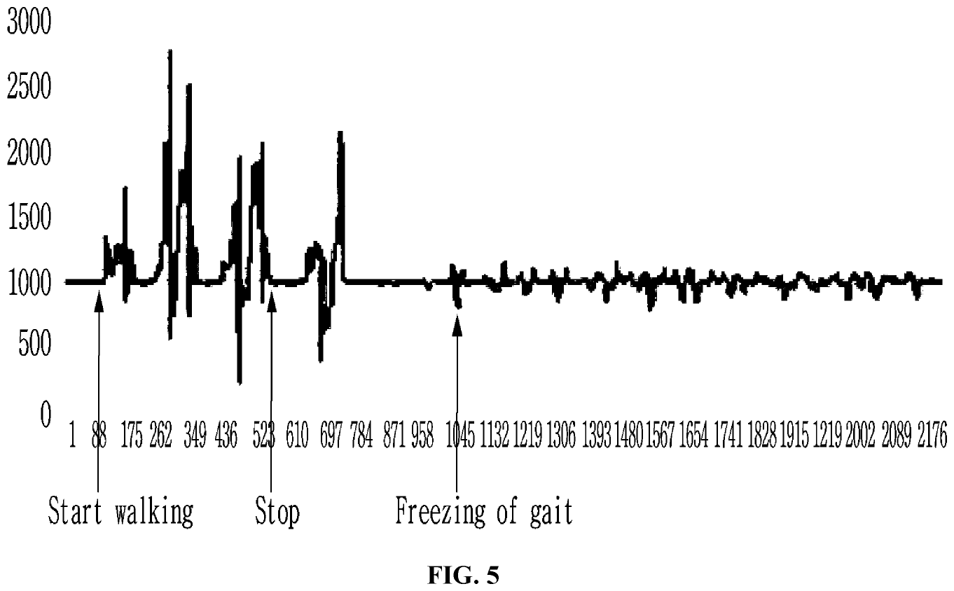

For example, a user may stop with both feet on the ground or with one foot separated from the ground. Here, as shown in FIG. 4, vibrations with amplitudes equal to or greater than a predetermined size may not be generated in a state where there is no walking. On the contrary, as shown in FIG. 5, vibrations with amplitudes equal to or greater than a predetermined size may be generated in a state where there is no walking. That is, in a state where the walking is stopped, vibrations may or may not be generated depending on whether freezing of gait occurs or not.

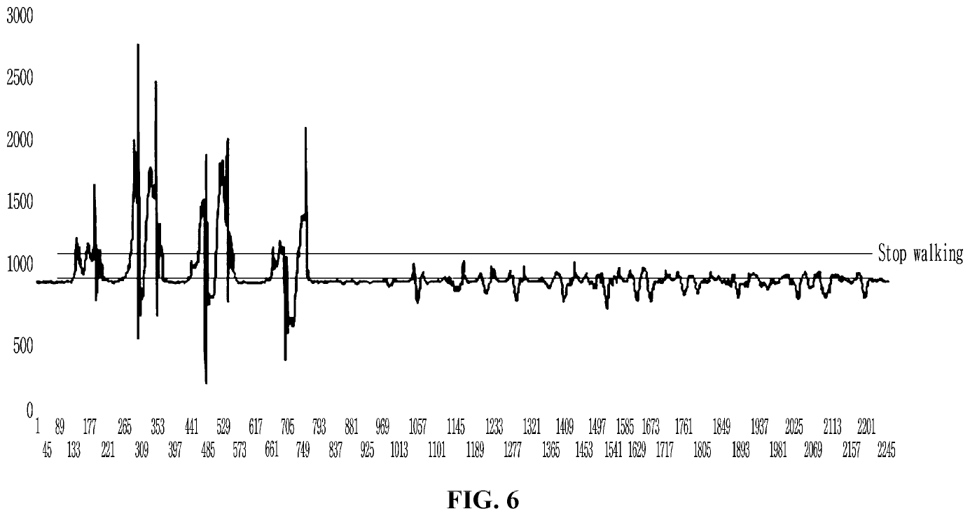

According to an example, when vibrations with amplitudes equal to or greater than a predetermined size are detected in a state where one foot of a user separates from the ground, the controller 130 may determine that freezing of gait occurs. That is, as shown in FIG. 6, when the amplitude of vibrations is equal to or smaller than a reference amplitude for stop, the controller 130 may determine that walking is stopped; when the amplitude of vibrations is equal to or greater than a reference amplitude for walking, the controller 130 may determine that walking is ongoing; and when the amplitude of vibrations is equal to or greater than the reference amplitude for stop and equal to or smaller than the reference amplitude for walking, the controller 130 may determine that freezing of gait occurs.

Specifically, as shown in FIG. 6, the controller 130 may predetermine a reference threshold value for walking and a reference threshold value for stop, and when a movement with a value between the two values lasts for a predetermined time, it may determine that freezing of gait occurs.

For example, the controller 130 may learn measurement values from the inertial measurement unit for a user's normal walking during a predetermined time. Based on this, it may determine the reference threshold value for walking and the reference threshold value for stop. While measuring vibration (movement) values generated during the user's walking, the controller 130 may predict whether freezing of gait occurs based on these reference threshold values using a pre-learned prediction model. Here, the prediction model may comprise a classification model which has undergone learning based on at least one of the size (degree) of a predetermined numerical value, the occurrence frequency of a predetermined numerical value, and a duration.

In order to enhance predictability for the occurrence of freezing of gait, the controller 130 may learn characteristics of vibration (movement) values during a predetermined time for different cases: a case when both of a user's feet are temporarily in contact with the ground while walking (Case 1), a case when a user stops in a normal way, such as while waiting for a traffic light (Case 2), and a case when a user undergoes freezing of gait, and may classify movements, with values between the reference threshold value for walking and the reference threshold value for stop, in the aforementioned three cases (Case 1, Case 2, Case 3). Based on the result of classification, the controller 130 may have the prediction model undergo reinforcement learning to enhance the accuracy in prediction of freezing of gait of each user.

In addition, the controller 130 may detect separation of a user's one foot from the ground using the pressure sensors, and when vibrations within a predetermined range are detected in this foot while it is separated from the ground, the controller 130 may predict that freezing of gait occurs.

According to an embodiment, when freezing of gait occurs, the controller 130 may control the vibration unit 120 such that one or more vibration generators generate vibrations. As an example, vibrations may be generated continuously or at predetermined time intervals. Furthermore, vibrations may be outputted in a predetermined pattern.

According to an embodiment, the controller 130 may calculate at least one of a stride and a step speed based on at least one of the size, period, tempo, direction, and duration of a movement, and when at least one of a stride and a step speed of a user is less than at least one of the average stride and the average step speed for a predetermined period of time, the controller 130 may determine it as a precursor state of freezing of gait and generate vibrations.

As an example, the controller 130 may predict that freezing of gait will occur based on measurement values of sensors. For example, when a stride of a user is narrower than the average of strides measured over a predetermined period of time by amount equal to or greater than a predetermined criterion, the controller 130 may predict that freezing of gait will occur. For another example, when a step speed is slower than the average of step speeds measured over a predetermined period of time by an amount equal to or greater than a predetermined criterion, the controller 130 may predict that freezing of gait will occur.

According to an example, when predicting that freezing of gait will occur, the controller 130 may proactively generate vibrations so that a user can focus on walking and generation of freezing of gait can be prevented through stimulation on its feet. When the stride or the step speed of the user enters back within an average range, the controller 130 may stop generating vibrations.

According to an example, the controller 130 may physically separate from the sensing unit 110 and the vibration unit 120. For example, the sensing unit 110 and the vibration unit 120 may be provided in an insole, whereas the controller 130 may be provided outside the insole. In this case, the sensing unit 110 and the vibration unit 120 may communicate with the controller 130 through wired or wireless communication methods.

According to an example, the controller 130 may communicate with an external user terminal (not shown). For this, the controller 130 may comprise a separate communication module.

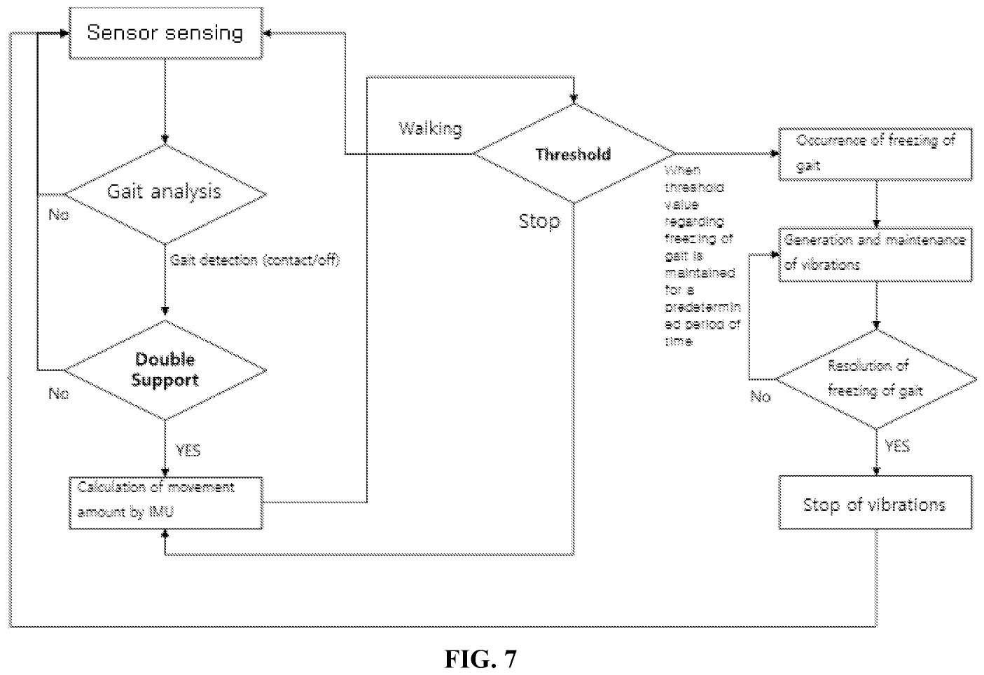

Referring to FIG. 7, the apparatus for forestalling and preventing freezing of gait may detect movements of a user using sensors and analyze the user's walking using measured sensor values. For example, the apparatus for forestalling and preventing freezing of gait may determine whether the user is walking or standing with both feet. Additionally, the apparatus for forestalling and preventing freezing of gait may calculate movements using sensor values of the inertial measurement unit; determine whether freezing of gait occurs based on a reference value regarding a predetermined movement; and generate vibrations based on the determination. Then, the apparatus for forestalling and preventing freezing of gait may determine whether the freezing of gait has been resolved and stop or continue generating vibrations.

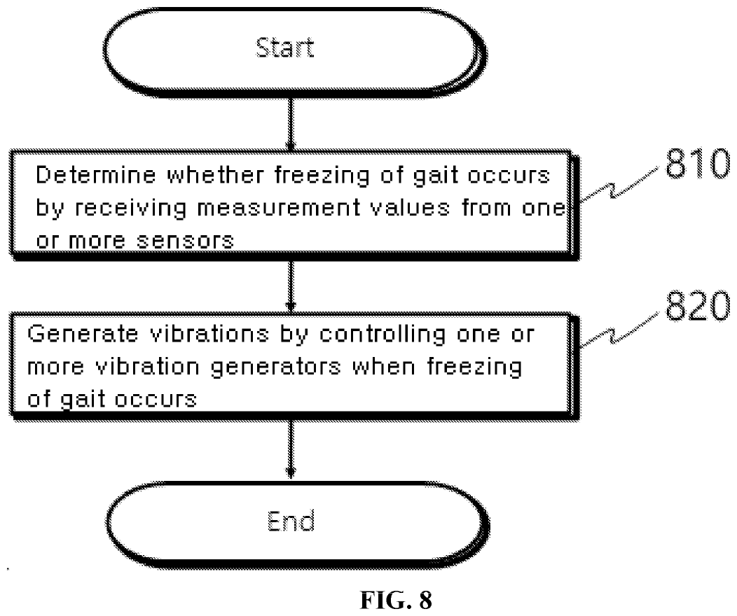

FIG. 8 is a flow diagram illustrating a method for forestalling and preventing freezing of gait according to an embodiment.

According to an embodiment, the apparatus for forestalling and preventing freezing of gait may be a computing device equipped with at least one processor and a memory for storing at least one program to be executed by the at least one processor.

According to an embodiment, the apparatus for forestalling and preventing freezing of gait may determine whether freezing of gait occurs by receiving measurement values from one or more sensors (810). As an example, a sensor may be at least one of a pressure sensor and an inertial measurement unit.

According to an example, the apparatus for forestalling and preventing freezing of gait may determine whether a user's feet are in contact with the ground of separate from the ground based on measurement values of pressure sensors. Additionally, the apparatus for forestalling and preventing freezing of gait may obtain movement information of a user's feet based on measurement values of the inertial measurement unit and determine freezing of gait based on at least one of the size, period, tempo, direction, and duration of a movement.

According to an example, the apparatus for forestalling and preventing freezing of gait may apply different criteria for determining whether freezing of gait occurs depending on whether both of a user's feet are in contact with the ground or one of the user's feet separates from the ground. For example, when both of a user's feet are in contact with the ground, the apparatus for forestalling and preventing freezing of gait may control one or more vibration generators to generate vibrations after a predetermined time has passed regardless of occurrence of the freezing of gait. Here, when the duration, during which both of the user's feet are in contact with the ground, has exceeded a predetermined time and vibrations have been generated, the apparatus for forestalling and preventing freezing of gait may determine whether to continue generating vibrations based on a specific motion of the user.

According to an example, when a specific motion of the user is detected after the duration, during which both of a user's feet are in contact with the ground, has exceeded a predetermined time and vibrations have been generated, the apparatus for forestalling and preventing freezing of gait may stop generating vibrations. When a specific motion of the user is not detected after the duration, during which both of a user's feet are in contact with the ground, has exceeded a predetermined time and vibrations have been generated, the apparatus for forestalling and preventing freezing of gait may continue generating vibrations.

According to an example, when one of a user's feet separates from the ground and at least one of the size, period, tempo, direction, and duration of a movement of the foot separating from the ground is within a predetermined range, the apparatus for forestalling and preventing freezing of gait may determine it as freezing of gait.

According to an embodiment, the apparatus for forestalling and preventing freezing of gait may control one or more vibration generators to generate vibrations when freezing of gait occurs (820).

The apparatus for forestalling and preventing freezing of gait may calculate at least of a stride and a step speed based on at least one of the size, period, tempo, direction, and duration of a movement. When at least one of the stride and step speed is less than at least one of an average stride and an average step speed during a predetermined period of time, the apparatus for forestalling and preventing freezing of gait may determine it as a precursor state of freezing of gait and generate vibrations.

Among embodiments regarding FIG. 8, embodiments overlapping with the embodiments described with reference to FIG. 1 to FIG. 7 are omitted.

An aspect of the present disclosure may be embodied as computer-readable codes in a recording medium readable by a computer. Codes and code segments for embodying the aforementioned program can easily be deduced by a computer programmer in the art. The computer-readable recording media may include all types of recording media storing data readable by a computer system. Examples of the computer-readable recording media may comprise ROMs, RAMs, CD-ROMs, magnetic tapes, floppy disks, optical disks, etc. In addition, the computer-readable recording media may be dispersed in computer systems connected with each other by network and be written and executed with computer-readable codes in a dispersed manner.

Thus far, the present disclosure is described with preferred embodiments. A person with ordinary knowledge in the technological field to which the present disclosure pertains would understand that the present disclosure can be embodied in modified forms without being beyond the essential characteristics. Therefore, it should be understood that the scope of the present disclosure is not limited to the above-described embodiments, but incorporates various embodiments within a scope equivalent to the contents described in the claims.

### LIST OF REFERENCE NUMBERS

100: Apparatus for forestalling and preventing freezing of gait
110: Sensing unit
120: Vibration unit
130: Controller

What is claimed is:

1. An apparatus for forestalling and preventing freezing of gait, comprising:
a sensing unit comprising at least one sensor;
a vibration unit comprising at least one vibration generator; and
a controller connected with the sensing unit and the vibration unit,
wherein the controller receives from the sensing unit measurement values of the at least one sensor to determine freezing of gait and controls the vibration unit to generate vibrations using the at least one vibration generator when it is determined as freezing of gait.

2. The apparatus for forestalling and preventing freezing of gait of claim 1, wherein the sensing unit comprises at least one of a pressure sensor and an inertial measurement unit.

3. The apparatus for forestalling and preventing freezing of gait of claim 2, wherein the controller determines based on measurement values of the pressure sensor whether a user's foot is in contact with the ground or separates from the ground.

4. The apparatus for forestalling and preventing freezing of gait of claim 3, wherein the controller obtains movement information of a user's foot based on measurement values of the inertial measurement unit and determine whether freezing of gait occurs based on at least one of the size, period, tempo, direction, and duration of a movement.

5. The apparatus for forestalling and preventing freezing of gait of claim 4, wherein the controller applies different criteria for determining whether freezing of gait occurs depending on whether both of a user's feet are in contact with the ground or one of the user's feet separates from the ground.

6. The apparatus for forestalling and preventing freezing of gait of claim 5, wherein, when both of a user's feet are in contact with the ground, the controller controls the vibration unit to generate vibrations after a predetermined time has passed regardless of determination of freezing of gait.

7. The apparatus for forestalling and preventing freezing of gait of claim 6, wherein the duration, during which both of a user's feet are in contact with the ground, has exceeded a predetermined time and vibrations have been generated, the controller determines whether to continue generating vibrations based on a specific motion of the user.

8. The apparatus for forestalling and preventing freezing of gait of claim 7, wherein,
when the specific motion of the user is detected after the duration, during which both of the user's feet are in contact with the ground, has exceeded a predetermined time and vibrations have been generated, the controller stops generating vibrations, and
when the specific motion of the user is not detected after the duration, during which both of the user's feet are in contact with the ground, has exceeded a predetermined time and vibrations have been generated, the controller continues generating vibrations.

9. The apparatus for forestalling and preventing freezing of gait of claim 5, wherein, when one of the user's feet separates from the ground and at least one of the size, period, tempo, direction, and duration of a movement of the foot separating from the ground is within a predetermined range, the controller determines it as freezing of gait.

10. The apparatus for forestalling and preventing freezing of gait of claim 4, wherein the controller calculates a stride or a step speed based on at least one of the size, period, tempo, direction, and duration of a movement, and when the stride or step speed is less than at least one of an average stride and an average step speed during a predetermined time, the controller determines it as a precursor state of freezing of gait and generate vibrations.

11. A method for forestalling and preventing freezing of gait, which is performed in a computing device equipped with at least one processor and a memory for storing at least one program to be executed by the at least one processor, comprising:
determining freezing of gait by receiving measurement values from at least one sensor; and
generating vibrations by controlling at least one vibration generator when it is determined as freezing of gait.

12. The method for forestalling and preventing freezing of gait of claim 11, wherein the sensor is at least one of a pressure sensor and an inertial measurement unit.

13. The method for forestalling and preventing freezing of gait of claim 12, wherein, in determining freezing of gait, whether a user's foot is in contact with the ground or separates from the ground is determined based on measurement values of the pressure sensor.

14. The method for forestalling and preventing freezing of gait of claim 13, wherein, in determining freezing of gait, movement information of a user's foot is obtained based on measurement values of the inertial measurement unit and whether freezing of gait occurs is determined based on at least one of the size, period, tempo, direction, and duration of a movement.

15. The method for forestalling and preventing freezing of gait of claim 14, wherein, in determining freezing of gait, different criteria for determining freezing of gait are applied depending on whether both of a user's feet are in contact with the ground or one of the user's feet separates from the ground.

16. The method for forestalling and preventing freezing of gait of claim 15, wherein, in generating vibrations, when both of a user's feet are in contact with the ground, vibrations are generated by controlling the at least one vibration generator after a predetermined time has passed regardless of determination of whether freezing of gait occurs.

17. The method for forestalling and preventing freezing of gait of claim 16, wherein, in generating vibrations, when the duration, during which both of the user's feet are in contact with the ground, has exceeded a predetermined time and vibrations have been generated, whether to continue generating vibrations is determined based on a specific motion of the user.

18. The method for forestalling and preventing freezing of gait of claim 17, wherein, in generating vibrations,
when the specific motion of the user is detected after the duration, during which both of the user's feet are in contact with the ground, has exceeded a predetermined time and vibrations have been generated, the vibrations stop being generated, and
when the specific motion of the user is not detected after the duration, during which both of the user's feet are in contact with the ground, has exceeded a predetermined time and vibrations have been generated, the vibrations continue being generated.

19. The method for forestalling and preventing freezing of gait of claim 15, wherein, in determining freezing of gait, when one of the user's feet separates from the ground and at least one of the size, period, tempo, direction, and duration of a movement of the foot separating from the ground is within a predetermined range, it is determined as freezing of gait.

20. The method for forestalling and preventing freezing of gait of claim 14, wherein, in determining freezing of gait, a stride or a step speed is calculated based on at least one of the size, period, tempo, direction, and duration of a movement, and when the stride or step speed is less than at least one of an average stride and an average step speed during a predetermined time, it is determined as a precursor state of freezing of gait and vibrations are generated.
